(19) United States
(12) Patent Application Publication
(10) Pub. No.: US 2013/0289291 A1
(43) Pub. Date: Oct. 31, 2013

(54) ESTOLIDE DERIVATIVES PREPARED FROM TRIGLYCERIDES

(71) Applicants: Sreejit Nair, Mumbai (IN); Saurabh Kaujalgikar, Dombivali (West) (IN); Daniele Vinci, Gent (BE)

(72) Inventors: Sreejit Nair, Mumbai (IN); Saurabh Kaujalgikar, Dombivali (West) (IN); Daniele Vinci, Gent (BE)

(73) Assignee: DOW GLOBAL TECHNOLOGIES LLC, Midland, MI (US)

(21) Appl. No.: 13/818,809

(22) PCT Filed: Sep. 20, 2011
(86) PCT No.: PCT/US11/52314

§ 371 Date: Feb. 25, 2013

(30) Foreign Application Priority Data
Sep. 24, 2010 (IN) 2807CHE2010

(51) Int. Cl. C07C 67/08

(52) U.S. Cl. CPC C07C 67/08
USPC 554/172; 554/227

(57) ABSTRACT

A process to prepare an estolide derivative composition comprises reacting a triglyceride having an unsaturation level of less than 0.1 m Eq/g and an alcohol having from 2 to 22 carbon atoms, under conditions such that a product including an oligomerized ester having residual hydroxyl groups is formed; and reacting the product with a capping agent under conditions such that an estolide derivative composition is formed. The compositions may exhibit desirably low pour point, desirable viscosity and thermoxidative stability, and a high level of renewable carbons, making them suitable for use as biolubricants, process fluids, plasticizers, or starting materials for polyurethane polymers.

### CROSS-REFERENCE TO RELATED APPLICATIONS

This application claims priority from provisional application serial number 2807/CHE/2010, filed Sep. 24, 2010, which is incorporated herein by reference in its entirety.

### BACKGROUND

1. Field of the Invention

This invention relates to the field of production of estolide derivatives. More particularly, it relates to processes for preparing estolide derivative compositions from triglycerides, wherein the compositions are useful as, for example, biolubricants.

2. Background of the Art

The lubricants (engine and non-engine) and process fluids industries today are searching for materials that are biodegradable. Biodegradability means that the lubricants and process fluids (hereinafter collectively “fluids”) degrade over a period of time, which may be measured by standard tests such as those promulgated by the European Organization of Economic Co-Operation and Development (OECD). These tests include OECD 301 B and OECD 301F. Recently, interest has been increasing in fluids which are not only biodegradable, but also renewable. Renewable products contain, by definition, high levels of renewable carbons, and standards are being set to encourage increasingly greater levels of renewability. For example, the European Ecolabel currently requires that hydraulic fluids must contain at least 50 weight percent (wt %) renewable carbons. Standards for determining levels of renewable carbons may be found in, for example, ASTM International (ASTM) D6866-08.

Researchers have attempted to meet requirements or recommendations for both biodegradability and renewability by including a variety of types of natural and synthesized oils in their fluids formulations. Unfortunately, many of these fluids exhibit pour points that are too high to enable use in certain important applications. The pour point is the lowest temperature at which the fluid will flow, and pour points below 0 degrees Celsius (° C.), desirably below −10° C., and more desirably below −15° C., are often necessary to ensure that the fluid remains useful in a given application's environment. These materials in many cases also suffer from poor thermoxidative stability at high temperatures (for example, above 90° C.), which may in some cases be due to the amount of unsaturation present in the acid fraction of their chemical structures. Desirable measurements of other properties, including but not limited to viscosity, may also be difficult to achieve.

In the quest to obtain these fluids properties, research has been done on estolides. Estolides are oligomeric fatty acids which may be formed by condensation of two or more fatty acid units to yield an ester linkage. Typically this condensation is accomplished by reacting a carboxylic acid moiety onto a double bond via acid catalysis.

An example of work on estolides may be found in U.S. Pat. No. 6,018,063 (Isbell, et al.), which relates to esters of estolides derived from oleic acids. That patent discloses synthesis of estolides involving homopolymerization of castor oil fatty acids or 12-hydroxystearic acid under thermal- or acid-catalyzed conditions.

Another example is U.S. Pat. No. 6,407,272 (Nelson, et al.). That patent teaches preparation of secondary alcohol esters of hydroxy acids (for example, ricinoleate esters of secondary alcohols). This is accomplished by reacting an ester of a hydroxy acid with a secondary alcohol in the presence of an organometallic transesterification catalyst.

Still another example may be found in Patent Cooperation Treaty Publication (WO) 2008/040864. That publication discloses a method for synthesizing estolide esters having a specified oligomerization level and a low residual acid index. The method involves simultaneous oligomerization of a saturated hydroxy acid and esterification of the hydroxyacid by a monoalcohol.

None of the above methods, however, has been shown to produce a fluid having desirable combinations of properties including high saturation level (unsaturation level that is less than 0.1 milliequivalents per gram (m Eq/g)), low pour point (at or below −10° C.), viscosity (less than 100 centipoise (cP) at 40° C., according to ASTM D445-94), and renewable carbons (at least 50 wt %). Thus, there is a need in the art for new fluids compositions meeting these requirements and/or new processes to produce appropriate compositions, such that they are capable of being used in lubricant applications where conditions reach particularly high and/or low temperatures.

### SUMMARY OF THE INVENTION

In one embodiment the invention provides a process to prepare an estolide derivative composition comprising: (1) reacting a triglyceride having an unsaturation level of less than 0.1 mEq/g and an alcohol having from 2 to 22 carbon atoms, under conditions such that a product including an oligomerized ester having residual hydroxyl groups is formed; and (2) reacting the product with an anhydride under conditions such that an estolide derivative composition is formed.

In another embodiment the invention provides a process to prepare an estolide derivative composition comprising: (a) reacting a triglyceride having an unsaturation level of less than 0.1 m Eq/g and excess alcohol having from 2 to 22 carbon atoms, in the presence of a Brønsted or Lewis acid or base catalyst; a tin-, titanium-, sodium-, or nitrogen-containing catalyst that is not a Brønsted or Lewis acid or base catalyst; or a combination thereof; under conditions such that a product including glycerol, excess alcohol, and an oligomerized ester having residual hydroxyl groups is formed; (b) separating the glycerol, excess alcohol, or both from the oligomerized ester; (c) reactively distilling the oligomerized ester to form a distilled oligomerized ester; (d) reacting the distilled oligomerized ester with a capping agent to cap the residual hydroxyl groups to form an estolide derivative; and (e) washing the estolide derivative to form an estolide derivative composition.

In still another embodiment the invention provides an estolide derivative composition prepared from a process comprising (1) reacting a triglyceride having an unsaturation level of less than 0.1 mEq/g and an alcohol having from 2 to 22 carbon atoms, under conditions such that a product including an oligomerized ester having residual hydroxyl groups is formed; and (2) reacting the product with an anhydride to form an estolide derivative composition.

### DETAILED DESCRIPTION OF THE EMBODIMENTS

The inventive process offers a convenient means to prepare estolide derivative compositions that, in certain embodiments, include a proportion of renewable carbons and exhibit as a property a pour point that is less than or equal to 0° C. In particular embodiments the proportion of renewable carbons is desirably at least 25 wt %, preferably at least 50 wt %, and the pour point is less than or equal to −10° C., preferably −15° C.

An estolide is a polyfunctional oligomer that contains ester linkages on the alkyl backbone of the molecule, and is formed by the esterification reaction between fatty acids. The invention includes a process wherein a starting triglyceride is concurrently both transesterified and oligomerized to form an estolide having residual hydroxyl groups, and the residual hydroxyl groups are then capped with a capping agent to form the estolide derivative composition.

The starting material for the process is at least one triglyceride (also called triacylglycerol, triacylglyceride, or “TAG”), or a combination of two or more TAGs. This TAG may be any compound defined as such, that is, an ester of glycerol bound to three fatty acid chains, which comprise the compound's acid fraction, which may contain three chains of the same fatty acid, or chains representing two or three different fatty acids. However, the selected TAG is one having an unsaturation that is preferably less than 0.1 m Eq/g, more preferably less than 0.05 m Eq/g, and most preferably less than 0.02 mEq/g. Thus, hydrogenated TAGs are particularly preferred. These TAGs preferably include an acid fraction wherein each carbon atom chain is, using conventional definitions, “long” (ranging from 13 to 21 carbon atoms), or “very long” (22 carbon atoms and greater), and in particular embodiments, contains from 14 to 23 carbon atoms. Particularly useful fatty acids for inclusion in the acid fraction chains are 12-hydroxy stearic acid, 12-hydroxy stearic acid ethyl ester (also called ethyl-12-hydroxy-stearate, or ethyl 12-HSA), and combinations thereof. These TAGs may be obtained from sources including, for example and not limited to, castor oil, cottonseed oil, lesquerella (bladderpod) oil, and ergot. For example, hydrogenated, castor oil, containing about 90 wt % of ethyl 12-HSA from ricinoleic acid, is preferred as a particularly convenient and economical source.

Preparation of the starting TAG, or TAGs, may be carried out by any known or conventional means and/or methods, most typically by hydrogenating a starting material, such as castor oil, or using a naturally hydrogenated hydroxytriglyceride. Another method involves reacting glycerol and a fatty acid, such as lauric, myristic, palmitic, stearic, or arachidic acid, or a combination thereof, which are saturated fatty acids. Other possible fatty acids may include, for example, unsaturated fatty acids, such as myristoleic, palmitoleic, sapienic, oleic, linoleic, α-linoleic, arachidonic, eicosapentaenoic, erucic, docosahexaenoic, and combinations thereof; however, those skilled in the art will recognize that the use of a highly saturated fatty acid will produce a TAG with lower unsaturation, and thus in preferred embodiments the use of saturated fatty acids, or fatty acids that are predominantly saturated, is preferred.

In certain embodiments the inventive process may be carried out as a process comprising two subprocesses. In the first subprocess (1), the selected TAG is transesterified through contact with a higher alcohol. The higher alcohol may contain from 2 to 22 carbon atoms, but should not contain just one carbon atom. Suitable examples may include, but are not limited to, 2-ethylhexanol, 2-(2-butoxy-propoxy)propan-1-ol (DPnB), 1-octanol, 2-octanol, and combinations thereof.

Under appropriate reaction conditions the reaction of the selected TAG and the higher alcohol results in an equilibrium product that includes some of the starting TAG as well as its corresponding estolide, which is at least partially oligomerized. By “oligomerized” it is meant that the resulting molecule has more than one repeating unit. Proportions of each will depend upon the process parameters, with the target endpoints being 100 percent (%) TAG conversion and the desired degree of oligomerization. In certain embodiments that degree of oligomerization may range, in non-limiting embodiments, from 1 to 4. Such determinative parameters may include, for example, the ratio of the starting materials, reaction time (which affects both conversion and degree of oligomerization), and temperature (which affects only degree of oligomerization). Thus, these aspects of the process may be conveniently controlled to optimize production as desired.

To accomplish subprocess (1), it is desirable that the mole ratio of the higher alcohol to TAG range from 2.8 to 8, preferably from 4 to 6.5, and more preferably from 5 to 6. It is also desirable that the temperature range from 85 degrees Celsius (° C.) to 250° C., preferably from 120° C. to 220° C., and most preferably from 150° C. to 180° C. Pressures may desirably range from 0.1 bar to 5 bar (10 kilopascals (kPa) to 500 kPa), preferably 0.5 bar to 4 bar (50 kPa to 400 kPa), with 1 to 3 bar (100 kPa to 300 kPa) being preferred to help maintain the alcohol in a liquid state at the reaction temperature. This helps to reduce evaporation losses, which in turn helps to ensure an optimum operating ratio is maintained. In one particular embodiment a combination of a mole ratio of the higher alcohol to TAG from 5 to 6; a temperature ranging from 150° C. to 180° C.; and a pressure ranging from 1 bar to 3 bar (100 kPa to 300 kPa); may be employed.

A suitable reactor vessel for (1) may be selected from continuous stirred tank reactors (CSTRs), batch stirred tank reactors, and semibatch stirred tank reactors. It is also effective to carry out this subprocess via reactive distillation.

An additional factor that may affect both conversion and degree of oligomerization within a given timeframe is that contact between the TAG and higher alcohol is desirably carried out in the presence of an acid or base catalyst. Suitable examples may include catalysts which are Brønsted or Lewis acids or bases; catalysts which are not Brønsted or Lewis acids or bases but are based on tin (Sn), titanium (Ti), sodium (Na), nitrogen (N) or a combination thereof; and combinations thereof. Thus, in some embodiments a given useful catalyst may be, for example, a Brønsted or Lewis acid or base and also be based on tin (Sn), titanium (Ti), sodium (Na), nitrogen (N) or a combination thereof. Specific examples of catalysts may include organotin compounds such as tin(II) octoate and dibutyltin dilaurate, which are Lewis acids; protonic acids such as sulfuric acid (H₂SO₄) and phosphoric acid (H₃PO₄); sodium methoxide (CH₃ONa), which is a Brønsted base; titanium(IV) chloride, which is a Lewis acid; di- and trimethylamine and propylamine; and combinations thereof. Preferably the catalyst is selected from Brønsted acids and bases, and more preferably from Brønsted bases. It is preferred that the catalyst be employed in this subprocess (1) in an amount ranging from 0.01 to 5 wt %; more preferably from 0.5 to 5 wt %; and most preferably from 1 to 2 wt %; based on the weight of the TAG.

Following the combined transesterification and oligomerization (1), the product of (1), i.e., the oligomerized estolide, may be further oligomerized in order to increase its molecular weight as desired. For convenience herein, this subprocess is termed (1a), and is optional in the inventive process, but may be preferred in certain embodiments. Such may be accomplished by means such as adding additional higher alcohol; increasing temperature; increasing time; removing a portion of the alcohol from the product of (1) at elevated temperature; reducing the partial pressure of alcohol by means such as, for example, applying vacuum, passing inert gas, or both; adding entrainers; and combinations thereof. Thus, a final molecular weight of the oligomerized estolide may in some embodiments range from 300 Daltons (Da) to 5,000 Da, and in preferred embodiments from 400 Da to 3,000 Da.

In a second subprocess (2), the oligomerized estolide is capped to form the final estolide derivative composition. This capping is accomplished by reaction of the oligomerized estolide, which contains residual hydroxyl groups from the TAG, and any compound that is capable of reacting with the residual hydroxyl group to form an ester, hereinafter referred to as a “capping agent.” Such capping agent may be selected from, in non-limiting example, anhydrides, organic carboxylic acids, and combinations thereof. Preferred among these are anhydrides, which may in certain embodiments accomplish the capping more rapidly and at a lower temperature than when capping is accomplished via esterification.

The capping may be carried out under a variety of conditions including, for example, at a molar ratio of the capping agent to the oligomerized estolide ranging from 0.5 to 3, preferably from 0.8 to 2, and more preferably from 1 to 1.5. It is desirable that the temperature range from 100° C. to 200° C., preferably from 110° C. to 140° C. and most preferably from 120° C. to 130° C. Pressures may desirably range from 1 bar to 2 bar (100 to 200 kPa), with 1 to 1.2 bar (100 to 120 kPa) being preferred to maintain the capping agent in liquid phase under the reaction conditions and thereby to reduce evaporation losses that would undesirably alter the operating ratio. In one particular embodiment it is desirable to employ a combination of a molar ratio of the capping agent to the oligomerized estolide ranging from 1 to 1.5; a temperature ranging from 120 to 130° C.; and a pressure ranging from 1 to 1.2 bar (100 to 120 kPa).

Suitable equipment for carrying out (2) may include a stirred tank reactor with overhead condenser. The reactor may be outfitted with a vacuum means for solvent removal. For ease of processing, however, it may in certain embodiments be particularly convenient to carry out the capping subprocess (2) in the same reactor vessel that is used for the combined transesterification/oligomerization (1) and/or the optional (1a) further oligomerization. Such selection may most conveniently facilitate accomplishing the inventive process as a batch, semi-continuous, or continuous process. In another embodiment (2) may be carried out in a separate reactor vessel. It is also possible to configure processing equipment such that at least a portion of the alcohol that may be removed following (1) and/or the optional (1a) may be recycled back to be used as a reactant in (1), regardless of whether the process is designed to be batch, semi-continuous or continuous.

An additional condition that may be optionally, but preferably, included among the reaction conditions for (2) is to conduct the contact between the oligomerized estolide and the capping agent in the presence of an acid or base catalyst. Suitable catalysts may be those based on or including tin (Sn), titanium (Ti), or nitrogen (N). These may include, for example, tin(II) dioctoate, titanium(IV) chloride, trimethylamine, and combinations thereof. Preferably the catalyst for (2) is tin(II) dioctoate. In amount it is preferred that the catalyst be present in an amount ranging from 0.01 to 5 wt %; more preferably from 0.5 to 5 wt %; and most preferably from 1 to 2 wt %; based on the weight of the oligomerized product used to begin (2).

In a particularly effective embodiment of the inventive process, it may be helpful to facilitate production of the target estolide derivative composition by adding or removing reactants and/or by-products. For example, subprocess (1) is an equilibrium reaction, but may be driven toward its transesterified and oligomerized product(s) by adding additional higher alcohol, removing some of the oligomerized estolide, for example, to a second reactor vessel, and/or removing any glycerol and/or excess alcohol from the (1) crude product. Subprocess (2), also an equilibrium reaction, produces the desired estolide derivative composition (and other products in crude mixture), but since it is desirable that the residual hydroxyl groups of the oligomerized ester are reacted quantitatively with the capping agent, removal of any excess capping agent from the crude product mixture may enable recovery of the target product estolide derivative composition without fractionation or other separation steps that may be difficult or expensive, and/or require additional equipment, to accomplish.

The overall inventive process may be summarized by the following formulaic process diagram:

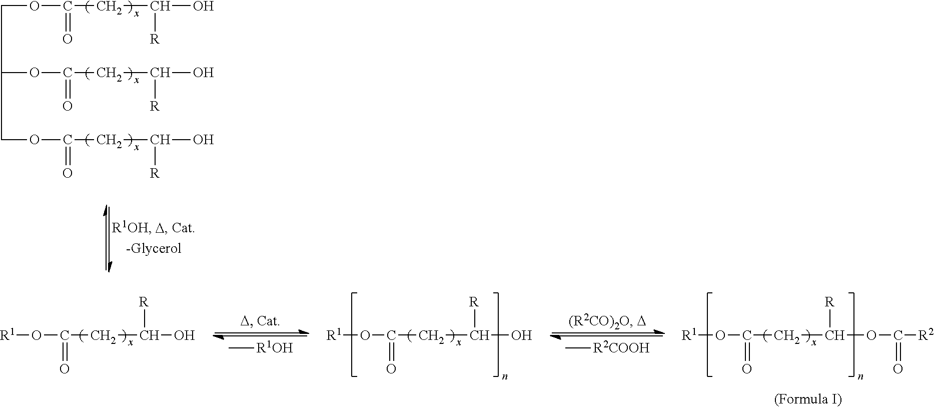

In the formulas hereinabove, O is oxygen, C is carbon, H is hydrogen, R is an alkyl group that contains from 6 to 12 carbon atoms, R¹ is an alkyl group that contains from 1 to 20 carbon atoms, R² is an alkyl group that contains from 1 to 11 carbon atoms, x is an integer ranging from 8 to 12, and n is an integer ranging from 1 to 20. The process diagram shows, in order from above left, then down, and to the right, subprocess (1), optional subprocess (1a), and subprocess (2), respectively.

The invention thus encompasses the estolide derivative compositions of Formula I, where Formula I is the last structure shown in the formulaic process diagram of paragraph [0027], as well as the process embodiments by which such compositions may be made. These estolide derivative compositions may have, as one primary advantage, methyl ester contents that are preferably less than 15 mole percent (mol %), more preferably less than 10 mol %, still more preferably less than 5 mol %, and most preferably less than 2 mol %. The compositions may also exhibit pour points that are preferably less than 0° C., more preferably less than −10° C., and most preferably less than −15° C. These compositions may also contain a proportion of carbon atoms that are renewable. Such proportion is preferably at least 25 wt %, and more preferably at least 50 wt %, based on the weight of the final product estolide derivative compositions.

Additional benefits derived from the inventive process are inventive compositions having high saturation level (unsaturation level that is less than 0.1 m Eq/g, preferably less than 0.05 m Eq/g, and more preferably less than 0.02 mEq/g, measured according to ASTM D5768-02 (2006), equivalent to an iodine number that is less than 0.3 g/100 g); low viscosity (less than 100 cP, preferably less than 50 cP, at 40° C., according to ASTM D445-94); and low acid numbers (preferably ranging from 0.5 to 0.01, more preferably from 0.3 to 0.01 mgKOH/g, according to ASTM D4662). The low unsaturation level may provide greater thermoxidative stability than that of materials having higher unsaturation levels. The estolide derivative compositions also have reduced residual functional groups in comparison with some estolides and estolide derivatives prepared via some other methods, due to the effect of the capping agent. Finally, yield of the final estolide derivative composition is preferably at least 78 wt %, more preferably at least 80 wt %, and most preferably at least 83 wt %, based on weight of the starting TAG.

In light of these properties, the final estolide derivative compositions may be useful in a wide range of applications. Such applications may include, in non-limiting examples, lubricants; process fluids; plasticizers for resins; power transmission fluids for hydraulics; heat transfer fluids; thickening agents; solvents; or surfactants. They may also be useful in the production of polyurethane polymer articles. Such polymers may be employed as foams, elastomers, coatings, or adhesives.

### EXAMPLES

### Example 1

Subprocess (1): An amount of hydrogenated castor oil (HCO) (100 grams, g) is charged to a 500-milliliter (mL) reactor. An amount of 2-ethylhexanol, 83.4 g, is added to maintain a mole ratio of HCO to 2-ethylhexanol of 1:6. Sodium methoxide powder (1.5 wt % in oil) (1.5 g) is added as a catalyst to the reaction mixture. The reaction is carried out at 150° C. for 4 hours (h). A nitrogen gas (N₂) blanket is maintained throughout the reaction in order to prevent moisture contact. At the end of the reaction, the product is cooled to 90° C. Concentrated phosphoric acid (H₃PO₄, 85 wt % solution) is added dropwise at 90° C. while stirring, in order to neutralize the sodium methoxide catalyst. A total of 6.9 g of the acid is added and the reaction product is maintained in liquid state at room temperature. The hot liquid product is then transferred to a separating funnel for washing. A total of 167 g of distilled water, at 80° C., is added in two steps for the washing. After each subprocess the bottom aqueous layer is separated under hot conditions and its pH is checked to ensure acid removal. The organic layer is then transferred to the reactor in order to remove excess 2-ethylhexanol. The 2-ethylhexanol is removed at 150° C. under vacuum (from 80 to 100 mbar, i.e., from 8 to 10 kPa). A total of 56.2 g of 2-ethylhexanol is removed from the product, and 114 g of first intermediate product is obtained.

Subprocess (1a): An amount of the first intermediate product from (1) hereinabove, 104 g, is charged to the 500 mL reactor. The reactor temperature is maintained at 120° C. for a few minutes to remove traces of water that may be present in the system. A total of 0.5 mol % of tin(II) octoate (Sn(C₈H₁₅O₂)₂) catalyst is added in this (1a). The reaction is carried out at 190° C. for 4 h, under vacuum (100 mbar, 10 kPa) to aid in removal of 2-ethylhexanol from the reactor during the reaction. A total of 9 g of 2-ethylhexanol is removed over the 4 h. A second intermediate product in the amount of 95 g is obtained. This subprocess increases the degree of oligomerization.

Subprocess (2): All of the second intermediate product (95 g) is charged to the 500 mL reactor. Isobutyric anhydride, 43.7 g, is added thereto. The reaction is carried out for 2 h at 120° C. At the end of the reaction excess isobutyric anhydride and isobutyric acid is removed under vacuum (100 mbar, 10 kPa) at 150° C. An amount of acid, 28.9 g, is collected, and 99.5 g of crude product is obtained.

The crude product from (2) is then transferred to a separating funnel for removal of tin (Sn) and acid. The product is washed with 25 g of 1 molar (M) sodium bicarbonate (NaHCO₃) solution at 60° C. Excess acid in the product is neutralized with carbon dioxide (CO₂) liberation from the crude product. The bottom aqueous layer is separated, and 100 g of hot distilled water is added stepwise for washing to remove salts formed during the neutralization. The product is then filtered through a bed of activated carbon (2 g), Celite (1 g) and magnesium sulfate (MgSO₄, 2 g). A total of 66 g of final estolide derivative composition product is obtained after filtration. Properties tested, standard methods employed, and test results are shown in Table 1.

TABLE 1

| Property | Measurement | ASTM Reference |
|---|---|---|
| Degree of | 1.62 | n/a* |
| Oligomerization¹ | | |
| Viscosity at 40° C., cP** | 41.5 | ASTM D445-94 |
| Viscosity at 100° C., cP | 8.5 | ASTM D445-94 |
| Acid Value, mgKOH/g | 1.37 | ASTM D4662 |
| Pour Point, ° C. | −19 | ASTM D97 |

*not applicable
**centipoise
¹number of repeating 12-hydroxystearoyl units, based on molecular weights (MW) derived from hydroxyl number (OH#)

### Example 2

Subprocess (1): The procedure of subprocess (1) in Example 1 is followed exactly, except that the reactor has a volume of 2,000 mL; the HCO amount is 765.0 g; the 2-ethylhexanol amount is 637.0 g; the sodium methoxide powder amount is 11.4 g; and the distilled water amount, used for washing, is 1.5 liter (L). A total of 360.7 g of 2-thylhexanol is removed from the product, and the first intermediate product total is 966.3 g.

Subprocess (1a): The procedure of subprocess (1a) in Example 1 is followed exactly, except that the reactor has a volume of 2,000 mL; the reaction is carried out under a vacuum of 20 mbar (2 kPa); a total of 76.6 g of 2-ethylhexanol is removed over 20 h of reaction; and the amount of second intermediate product is 822.5 g.

Subprocess (2): The procedure of subprocess (2) in Example 1 is followed exactly, except that the reactor has a volume of 2,000 mL; the amount of isobutyric anhydride is 188.5 g; a vacuum of 20 mbar (2 kPa) is used for excess isobutyric anhydride and isobuturic acid removal; 131.6 g of acid is collected; and 808.0 g of crude final product is obtained.

Product washing and filtration are carried out as in Example 1, except that a volume of 1 M NaHCO₃ solution is used for washing, and a total of 660 g of estolide derivative composition product is obtained. Results of properties testing on the final products, done using ASTM standard methods as referenced in Table 1, are shown in Table 2.

TABLE 2

| Property | Measurement |
|---|---|
| Degree of Oligomerization | 2.18 |
| Viscosity at 40° C., cP* | 46.1 |
| Viscosity at 100° C., cP* | 8.73 |
| Acid Value, mgKOH/g | 0.06 |
| Pour Point, ° C. | −18 |

*cP = centipoise

### Examples 3-6

Subprocess (1): The procedure of subprocess (1) of Example 1 is carried out, except that the amount of HCO is 440 g; the amount of 2-ethylhexanol is 333.6 g; and the amount of sodium methoxide is 6 g. The amount of first intermediate product of this subprocess (1) is 458.6 g.

Subprocess (1a): The procedure of subprocess (1a) of Example 1 is followed, except that varying amounts of first intermediate product (of (1)) are employed and the extent of vacuum and the reaction times are varied to obtain varying degrees of oligomerization in the second intermediate products. These samples are designated as Examples 3-5. One product sample from (1) is not subjected to this (1a) and thus exhibits only the degree of oligomerization achieved during (1). This sample is designated as Example 6.

Subprocess (2): The procedure of subprocess (2) of Example 1 is followed, except that the procedure is applied to each of the (second intermediate) products of subprocess (1a) (i.e., Examples 3-5), and also to the (first intermediate) product sample from (1) that is not subjected to (1a) (i.e., Example 6). Each of the (2) crude samples is washed and filtered as in previous Examples to obtain final estolide derivative composition products.

Proton nuclear magnetic resonance (H¹-NMR) analysis is done on each sample to determine the degree of oligomerization. Testing is also done by gel permeation chromatography-mass spectrometry (GPC-MS) to determine the molecular weight (MW) distribution in Daltons (Da) following (1a) (for Examples 3-5) and (2) (for Examples 3-6). The results, as well as viscosity testing according to ASTM D445-94, are shown in Table 3.

TABLE 3

| | Example Number | | | | | | |
|---|---|---|---|---|---|---|---|
| | Example 3 | | Example 4 | | Example 5 | | Example 6 |
| | (1a) | (2) | (1a) | (2) | (1a) | (2) | (2) (no (1a)) |
| Subprocess completed | | | | | | | |
| Product of subprocess (1) (g) | 50 | n/a | 50 | n/a | 100 | n/a | 65 |
| Catalyst (g) | 0.38 | n/a | 0.44 | n/a | 0.76 | n/a | n/a |
| Reaction time (h) | 3 | 2 | 4 | 2 | 4 | 2 | 2 |
| 2-ethylhexanol collected (g) | 4.9 | n/a | 6.1 | n/a | 3.6 | n/a | n/a |
| Isobutyric anhydride (g) | n/a | 9.7 | n/a | 9.6 | n/a | 45 | 23 |
| Degree of oligomerization | 1.69 | n/a | 2.36 | n/a | 1.58 | n/a | 1.26 |
| GPC-MS (area %) | | | | | | | |
| <500 Da | 68.34 | 38.55 | 21.77 | 22.34 | n/a | n/a | n/a |
| 500~1000 Da | 23.48 | 28.97 | 23.39 | 23.36 | n/a | n/a | n/a |
| 1000~1500 Da | 8.18 | 25.97 | 32.12 | 32.12 | n/a | n/a | n/a |
| >1500 Da | 0 | 0 | 22.18 | 22.18 | n/a | n/a | n/a |
| Viscosity (cP) at 40° C. | n/a | 43.5 | n/a | 74.5 | n/a | 34 | 26.5 |
| Viscosity (cP) at 100° C. | n/a | 9.5 | n/a | 12.5 | n/a | 9 | 5.5 |

n/a not applicable or not tested
Da = Daltons
< means “less than”
~ means “approximately”
> means “greater than”

### Examples 7-11

A series of experiments is carried out to demonstrate the relationship of the alcohol to TAG ratio and the degree of oligomerization and conversion accomplished as a result of (1). Procedures to prepare the subprocess (1) (first intermediate) products are carried out as in Example 1, except that the molar ratios of alcohol to TAG are varied as shown in Table 4, and all reactions are carried out for 4 h at a temperature of 190° C. Results of analysis by H¹-NMR show amounts of dimer and unreacted diglycerides and monoglycerides in the (1) (first intermediate) product.

TABLE 4

| | Exam- | Exam- | Exam- | Exam- | Example |
|---|---|---|---|---|---|
| | ple 7 | ple 8 | ple 9 | ple 10 | 11 |
| Hydrogenated castor | 100 | 100 | 100 | 100 | 50 |
| oil (HCO) (g) | | | | | |
| Temperature (° C.) | 190 | 190 | 190 | 190 | 190 |
| Catalyst wt % of HCO | 2.5 | 1.5 | 2 | 2.5 | 1.5 |
| Reaction time (h) | 4 | 4 | 4 | 4 | 4 |
| Mole ratio of | 6 | 5 | 4 | 3 | 2.8 |
| 2-ethylhexanol:HCO | | | | | |
| % dimer | 22 | 31 | 40 | 62 | 61 |
| Degree of | 1.22 | 1.31 | 1.40 | 1.62 | 1.61 |
| oligomerization | | | | | |
| Unreacted diglycerides | <5% | ~11% | ~23% | ~29% | ~25% |
| and monoglycerides | | | | | |

### Examples 12-15

A series of experiments is carried out to demonstrate the effect of temperature on the degree of oligomerization and conversion accomplished as a result of subprocess (1). Procedures to prepare the (1) (first intermediate) products are carried out as in Example 1, except that temperatures are varied from 100 to 190° C. as shown in Table 5. All reactions are conducted at a mole ratio of alcohol to TAG of 6:1. Results of analysis by H¹-NMR show amounts of dimer and unreacted diglycerides and monoglycerides in the (1) (first intermediate) product.

TABLE 5

| | Example | Example | Example | Example |
|---|---|---|---|---|
| | 12 | 13 | 14 | 15 |
| Hydrogenated castor oil | 100 | 100 | 50 | 50 |
| (HCO) (g) | | | | |
| Temperature (° C.) | 190 | 150 | 120 | 100 |
| Catalyst, based on wt % | 2.5 | 1.5 | 1.5 | 1.5 |
| HCO | | | | |
| Reaction time (h) | 4 | 4 | 4 | 4 |
| Mole ratio of | 6 | 6 | 6 | 6 |
| 2-ethylhexanol to HCO | | | | |
| Degree of | 1.26 | 1.26 | 1.1 | 1.06 |
| oligomerization | | | | |
| Unreacted diglycerides | <5% | <5% | <5% | <5% |
| and monoglycerides | | | | |

< means “less than”

### Comparative Example A

Thermoxidative stability is tested according to ASTM D-2893B. In this method, a 300-mL sample of each composition is placed into a borosilicate glass tube and heated to 121° C. in dry air for 312 hours (13 days). The viscosity of the fluid at 40° C. and at 100° C. before and after the test is measured according to ASTM D7042, and lower percentages of change indicate improvements in thermoxidative stability.

The testing is carried out using, as described below, a sample prepared as described in Example 2. That sample, an estolide derivative composition of the invention, has the following physical properties: kinetic viscosity at 40° C. (KV40)=46.1 centistokes (cSt); kinetic viscosity at 100° C. (KV100)=8.73 cSt; pour point=−18° C.; unsaturation level=0.01 mEq/g. The comparison includes four materials, as follows:

1. Formulated Estolide Derivative Composition—This lubricant contains an Example 2 sample (98.5%), plus Irganox™ L57 (0.5%) and Irganox™ L101 (1.0%). The Irganox™ products are antioxidants and are available from BASF. The formulation is prepared by adding the antioxidants to the base oil (the Example 2 sample) and stirring at 60° C. until they are dissolved. Formulated Estolide Derivative Composition has a KV40=47.0 cSt; KV100=8.8 cSt; and a pour point of −18° C.
2. Plantohyd™ 40 N—A formulated bio-hydraulic fluid from Fuchs. The primary base oil in the formulation is rapeseed oil. The product contains an additive package. It has the following properties: KV40=44.5 cSt; KV100=9.7 cSt; VI=212; pour point=−30° C.
3. Eco-Hyd™ 68S—A formulated bio-hydraulic fluid from Fuchs Lubritech. The primary base oil in the formulation is a synthetic ester. The product contains an additive package. It has the following properties: KV40=64.4 cSt; KV100=11.7 cSt; VI=179; pour point=−39° C.
4. Novus™100—A formulated bio-hydraulic fluid from Chemtool Incorporated. The primary base oil in the formulation is a canola oil and the product contains an additive package. It has the following properties: KV40=45.6 cSt; KV100=10.2 cSt; Viscosity index (VI)=222.

Results of the tests are shown in Tables 6 and 7. This comparison illustrates the dramatic improvement in thermoxidative stability achieved by the inventive composition. No viscosity data is obtained for the Novus™ 100 fluid at 100° C., in view of its viscosity performance at 40° C.

TABLE 6
Kinetic Viscosity at 100° C.

| | Initial | | |
|---|---|---|---|
| | Viscosity, | Final Viscosity, | |
| | cSt | cSt | % Change in |
| days | 0 days | 13 days | viscosity |
| Formulated Example 2 | 8.8 | 9.0 | 2.2 |
| Planto hyd 40N | 9.7 | [solidified] | >100 |
| Eco-Hyd 68S | 11.7 | 56.3 | 481 |

TABLE 7
Kinetic Viscosity at 40° C.

| | Initial | | |
|---|---|---|---|
| | Viscosity, | Final | |
| | cSt | Viscosity, cSt | % Change |
| days | 0 days | 13 days | in viscosity |
| Formulated Example 2 | 47.0 | 50.0 | 6.3 |
| Plantohyd 40N | 44.5 | [solidified] | >100 |
| Eco-Hyd 68S | 64.4 | 692 | 975 |
| Novus 100 | 45.2 | 83.1 (note 1) | 82 (note 1) |

(note 1) - Test abandoned after 11 days due to significant viscosity increase

1. A process to prepare an estolide derivative composition comprising:
(1) reacting a triglyceride having an unsaturation level of less than 0.1 mEq/g and an alcohol having from 2 to 22 carbon atoms, under conditions such that a product including an oligomerized ester having residual hydroxyl groups is formed; and
(2) reacting the product with a capping agent under conditions such that an estolide derivative composition is formed.

2. The process of claim 1 wherein the triglyceride includes acid fraction chains that each contain from 14 to 23 carbon atoms.

3. The process of claim 1 wherein the triglyceride is hydrogenated castor oil.

4. The process of claim 1 wherein the alcohol is selected from the group consisting of 2-ethylhexanol, 2-(2-butoxy-propoxy)propan-1-ol, 1-octanol, 2-octanol, and combinations thereof.

5. The process of claim 1 wherein the conditions of (1) include at least one of: (a) a mole ratio of the alcohol to the triglyceride ranging from 2.8 to 8; (b) a temperature ranging from 85° C. to 250° C.; (c) a pressure ranging from 100 kPa to 500 kPa; and (d) a combination thereof.

6. The process of claim 1 wherein the conditions include at least one of: (a) a mole ratio of the alcohol to the triglyceride ranging from 4 to 6.5; (b) a temperature ranging from 120° C. to 220° C.; (c) a pressure ranging from 100 kPa to 300 kPa; and (d) a combination thereof.

7. The process of claim 1 wherein the conditions of (1) include using a catalyst selected from the group consisting of (a) catalysts that are Brønsted and Lewis acids and bases; (b) catalysts that contain tin, titanium, sodium, or nitrogen and are not Brønsted or Lewis acids or bases; and (c) a combination thereof.

8. The process of claim 1 further including (1a), wherein the product of (1) is further oligomerized, prior to (2), by a means selected from:
(i) adding an additional amount of an alcohol having from 2 to 22 carbon atoms;
(ii) subjecting the product of (1) to a higher temperature;
(iii) allowing additional reaction time;
(iv) removing alcohol present in the product;
(iv) reducing the alcohol's partial pressure; and
(v) combinations thereof;
to form the product that is used in (2).

9. The process of claim 1 wherein (2) includes using a catalyst selected from the group consisting of tin-, titanium-, and nitrogen-containing catalysts and combinations thereof.

10. The process of claim 1 wherein the product containing an oligomerized ester that is used in (2) has a molecular weight ranging from 300 to 5,000 Daltons (Da).

11. The process of claim 1 wherein the capping agent is selected from the group consisting of anhydrides, organic carboxylic acids, and combinations thereof.

12. The process of claim 1 wherein the conditions of (2) include at least one of: (a) a molar ratio of the capping agent to the oligomerized ester ranging from 0.5 to 3; (b) a temperature ranging from 100° C. to 200° C.; (c) a pressure ranging from 100 to 200 kPa; and (d) a combination thereof.

13. The process of claim 1 wherein the estolide derivative composition exhibits at least one property selected from: (a) a methyl ester content less than 2 mol %; (b) a pour point less than −15° C.; (c) a renewable carbon content of at least 50 weight percent; (d) an iodine number less than 0.3 g/100 g; (e) an unsaturation level less than 0.1 mEq/g; and (f) a combination thereof.

14. A process to prepare an estolide derivative composition comprising: (a) reacting a triglyceride having an unsaturation level of less than 0.1 mEq/g and excess alcohol having from 2 to 22 carbon atoms, in the presence of a catalyst that is selected from Brønsted and Lewis acids and bases; catalysts that contain tin-, titanium-, sodium-, or nitrogen and are not Brønsted or Lewis acids or bases; and combinations thereof; under conditions such that a product including glycerol, excess alcohol, and an oligomerized ester having residual hydroxyl groups is formed; (b) separating the glycerol, excess alcohol, or both from the oligomerized ester; (c) reactively distilling the oligomerized ester to form a distilled oligomerized ester; (d) reacting the distilled oligomerized ester with a capping agent to cap the residual hydroxyl groups to form a crude estolide derivative product; and (e) washing the crude estolide derivative product to form an estolide derivative composition.

15. An estolide derivative composition prepared by the process of claim 1, wherein the composition is a lubricant, a process fluid, a plasticizer, a power transmission fluid, a heat transfer fluid, a thickening agent, a solvent, a surfactant, or a combination thereof.
